(19) United States
(12) Patent Application Publication
(10) Pub. No.: US 2023/0060521 A1
(43) Pub. Date: Mar. 2, 2023

(54) PROFICIENCY DETERMINATION APPARATUS, METHOD, AND NON TRANSITORY COMPUTER READABLE MEDIUM

(71) Applicant: KABUSHIKI KAISHA TOSHIBA, Tokyo (JP)

(72) Inventors: Izumi FUKUNAGA, Tokyo (JP); Yasunobu YAMAUCHI, Yokohama (JP); Akiyuki TANIZAWA, Kawasaki (JP); Tsukasa IKE, Tokyo (JP)

(73) Assignee: KABUSHIKI KAISHA TOSHIBA, Tokyo (JP)

(21) Appl. No.: 17/652,189

(22) Filed: Feb. 23, 2022

(30) Foreign Application Priority Data
Sep. 1, 2021 (JP) 2021-142615

(51) Int. Cl. G06Q 10/06; A61B 5/00; A61B 5/16

(52) U.S. Cl. CPC G06Q 10/063114; A61B 5/165; A61B 5/7275; G06Q 10/06395

(57) ABSTRACT

According to one embodiment, a proficiency determination apparatus includes a processing circuit. The processing circuit acquires first time-series data about biological information of a worker in a predetermined period. The processing circuit calculates second time-series data about a physiological index indicating a mental stress on the worker by analyzing the first time-series data. The processing circuit calculates an appearance condition of the physiological index by analyzing the second time-series data. The processing circuit determines a proficiency of the worker based on the appearance condition of the physiological index.

### CROSS-REFERENCE TO RELATED APPLICATIONS

This application is based upon and claims the benefit of priority from Japanese Pat. Application No. 2021-142615, filed Sep. 1, 2021, the entire contents of which are incorporated herein by reference.

### FIELD

Embodiments described herein relate generally to a proficiency determination apparatus, a method, and a non-transitory computer readable medium.

### BACKGROUND

Determining the proficiency of a worker engaged in a technical work in a factory or the like is important to perform training or education necessary to a worker (beginner) having a low proficiency. For example, there is a method of determining the proficiency of a worker based on the working time required for the worker or the action or posture of the worker during the work.

The ability to evaluate the quality of a product (to be called “quality evaluation ability” hereinafter) is an example of the ability that a worker should acquire. When a worker performs a work by paying attention to the quality of a product, the quality of the product to be shipped from a factory or the like probably improves. Accordingly, it is desirable to determine the proficiency about the quality evaluation ability of a worker. However, this ability depends on the subjective recognition of a worker. This makes it difficult to measure the degree of this ability based on objective information such as the working time, the action, or the posture described above.

### BRIEF DESCRIPTION OF THE DRAWINGS

FIG. 1 is a block diagram showing a configuration example of a proficiency determination apparatus according to the first embodiment;

FIG. 2 is a flowchart showing an operation example of the proficiency determination apparatus according to the first embodiment;

FIG. 3 is a view showing an example of time-series data of an LF/HF ratio according to the first embodiment;

FIG. 4 is a view showing an example of the frequency of a local maximum in the time-series data of the LF/HF ratio according to the first embodiment;

FIG. 5 is a view showing an example of the correspondence between the frequency of the local maximum and the proficiency in the time-series data of the LF/HF ratio according to the first embodiment;

FIG. 6 is a block diagram showing a configuration example of a proficiency determination apparatus according to the second embodiment;

FIG. 7 is a view showing an example of the working condition of a worker corresponding to time-series data of an LF/HF ratio according to the second embodiment;

FIG. 8 is a view showing the appearance characteristic of a physiological index corresponding to the working condition of a worker;

FIG. 9 is a view showing an example of the frequency of a local maximum in the time-series data of the LF/HF ratio according to the second embodiment;

FIG. 10 is a view showing an example of the correspondence between the frequency of the local maximum and the proficiency in the time-series data of the LF/HF ratio according to the second embodiment;

FIG. 11 is a block diagram showing a configuration example of a proficiency determination apparatus according to the third embodiment;

FIG. 12 is a view showing an example of the correspondence between a working zone and product quality information in time-series data of an LF/HF ratio according to the third embodiment;

FIG. 13 is a view showing the appearance characteristic of a physiological index corresponding to the product quality information according to the third embodiment;

FIG. 14 is a view showing an analytical example of the frequency of a local maximum in the time-series data of the LF/HF ratio according to the third embodiment;

FIG. 15 is a view showing an analytical example of the frequency of a local maximum in the time-series data of the LF/HF ratio according to the third embodiment;

FIG. 16 is a block diagram showing a configuration example of a proficiency determination apparatus according to the fourth embodiment;

FIG. 17 is a view showing an example of display data according to the fourth embodiment;

FIG. 18 is a view showing an example of display data according to the fourth embodiment;

FIG. 19 is a block diagram showing a configuration example of a proficiency determination apparatus according to the fifth embodiment;

FIG. 20 is a view showing an example of improvement proposal information according to the fifth embodiment; and

FIG. 21 is a block diagram showing a configuration example of hardware and software of the proficiency determination apparatuses according to the first to fifth embodiments.

### DETAILED DESCRIPTION

In general, according to one embodiment, a proficiency determination apparatus includes a processing circuit. The processing circuit acquires first time-series data about biological information of a worker in a predetermined period. The processing circuit calculates second time-series data about a physiological index indicating a mental stress on the worker by analyzing the first time-series data. The processing circuit calculates an appearance condition of the physiological index by analyzing the second time-series data. The processing circuit determines a proficiency of the worker based on the appearance condition of the physiological index.

A proficiency determination apparatus, a method, and a non-transitory computer readable medium according to embodiments will be explained below with reference to the accompanying drawings. In the following embodiments, parts denoted by the same reference numerals perform similar operations, so a repetitive explanation thereof will be omitted as needed.

### First Embodiment

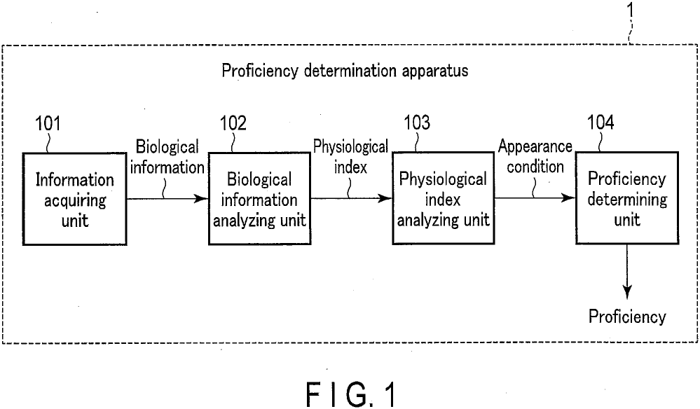

FIG. 1 is a block diagram showing a configuration example of a proficiency determination apparatus 1 according to the first embodiment. The proficiency determination apparatus 1 is an apparatus for determining the proficiency about the quality evaluation ability of a worker. The proficiency determination apparatus 1 includes an information acquiring unit 101, a biological information analyzing unit 102, a physiological index analyzing unit 103, and a proficiency determining unit 104. The information acquiring unit 101, the biological information analyzing unit 102, the physiological index analyzing unit 103, and the proficiency determining unit 104 are respectively examples of a first acquiring unit, a first analyzing unit, a second analyzing unit, and a determining unit.

The information acquiring unit 101 acquires biological information of a worker. More specifically, the information acquiring unit 101 acquires first time-series data about biological information of a worker in a predetermined period. The biological information is information about, e.g., the NN interval, the electrodermal activity, the perspiration, the blood flow, the body temperature, or the brain wave. These pieces of information can also be used as physiological indices. In this embodiment, it is assumed that the biological information is information about the NN interval. Subsequently, the information acquiring unit 101 outputs first time-series data about the acquired biological information to the biological information analyzing unit 102.

The biological information analyzing unit 102 analyzes the biological information of a worker. More specifically, the biological information analyzing unit 102 analyzes the first time-series data and calculates second time-series data about a physiological index indicating the mental stress on the worker. Examples of the physiological index are an LF/HF (Low Frequency/High Frequency) ratio, an LF (Low Frequency) value, an HF (High Frequency) value, a VLF (Very Low Frequency) value, a total power value, an HR (Heart Rate) value, a Mean NN (Mean of the NN intervals) value, an SDNN (Standard Deviation of the NN intervals) value, an RMSSD (Root Mean Square of Successive Differences) value, an NN50 (the total number of heartbeats for which the successive differences exceed 50 ms) value, a pNN50 (the ratio of heartbeats for which the successive differences exceed 50 ms) value, or a CVRR (Coefficient of Variation of R-R Interval) value related to the NN interval. In this embodiment, it is assumed that the physiological index is the LF/HF ratio. Subsequently, the biological information analyzing unit 102 outputs the calculated second time-series data about the physiological index to the physiological index analyzing unit 103.

The physiological index analyzing unit 103 analyzes the physiological index of the worker. More specifically, the physiological index analyzing unit 103 analyzes the second time-series data and calculates the appearance condition of the physiological index. For example, the appearance condition of the physiological index is the frequency or magnitude of a parameter including a local maximum, a local minimum, a mean, a dispersion, a fluctuation, a global maximum, a global minimum, a differential value, and an integral value related to the physiological index. In this embodiment, it is assumed that the appearance condition of the physiological index is the frequency of a local maximum about the LF/HF ratio. Subsequently, the physiological index analyzing unit 103 outputs the calculated appearance condition of the physiological index to the proficiency determining unit 104.

The proficiency determining unit 104 determines the proficiency of the worker. More specifically, the proficiency determining unit 104 determines the proficiency of the worker based on the appearance condition of the physiological index. For example, the proficiency determining unit 104 determines the proficiency of the worker based on whether the frequency of a local maximum about the physiological index is equal to or larger than a threshold. In this embodiment, the proficiency determining unit 104 determines the proficiency of the worker based on whether the frequency of a local maximum about the LF/HF ratio is equal to or larger than a threshold. Subsequently, the proficiency determining unit 104 outputs the determined proficiency of the worker to the inside or the outside of the proficiency determination apparatus 1.

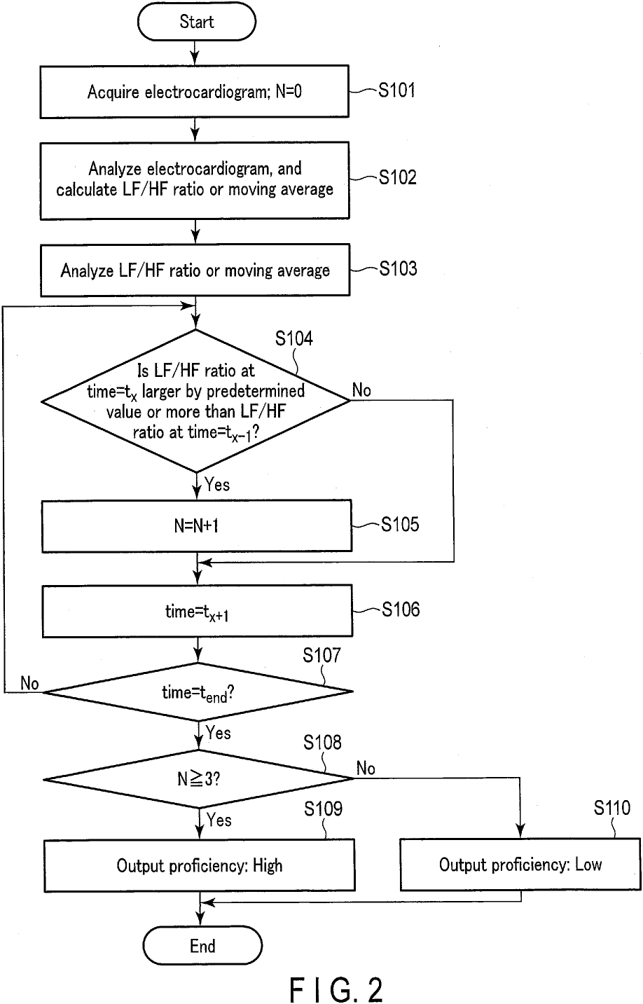

FIG. 2 is a flowchart showing an operation example of the proficiency determination apparatus 1 according to the first embodiment. In step S101, the proficiency determination apparatus 1 acquires an electrocardiogram by using the information acquiring unit 101. An electrocardiogram is an example of the biological information about the NN interval of a worker, and acquired from, e.g., a biological sensor placed on the body surface of the worker. The electrocardiogram need only be time-series data over a period or a time length during which the LF/HF ratio can be calculated. At the same time, the proficiency determination apparatus 1 sets a frequency N (N is an integer) of the physiological index at 0 (N = 0) .

In step S102, the proficiency determination apparatus 1 analyzes the electrocardiogram by using the biological information analyzing unit 102, and calculates the LF/HF ratio or the moving average. For example, the proficiency determination apparatus 1 calculates time-series data of a change in NN interval by analyzing the acquired electrocardiogram, and calculates a power spectrum about the change in NN interval by applying Fourier transform or an autoregressive model to the time-series data. Subsequently, the proficiency determination apparatus 1 calculates a frequency component (LF value) in an LF (Low Frequency) band (0.004 to 0.15 Hz) of the power spectrum, and a frequency component (HF value) in an HF (High Frequency) band (0.15 to 0.4 Hz) thereof. Finally, the proficiency determination apparatus 1 calculates the ratio (LF/HF ratio) of the calculated low-frequency and high-frequency components. That is, the proficiency determination apparatus 1 need only use the existing analyzing method for calculating the LF/HF ratio from the electrocardiogram. Note that it is also possible to calculate a frequency component (VLF value) in a VLF (Very Low Frequency) band (0.0033 to 0.04 Hz) of the calculated power spectrum, or a frequency component (total power value) in a total frequency band (0 to 0.04 Hz) thereof.

The LF/HF ratio is an example of the physiological index indicating the mental stress (stress) on the worker. Generally, the LF/HF ratio is regarded as an index indicating the balance between the activity of the sympathetic nerve and the activity of the parasympathetic nerve. In this embodiment, it is assumed that the higher the LF/HF ratio of the worker, the stronger the stress on the worker. On the other hand, it is assumed that the worker has almost no stress or is relaxed when the LF/HF ratio of the worker is low.

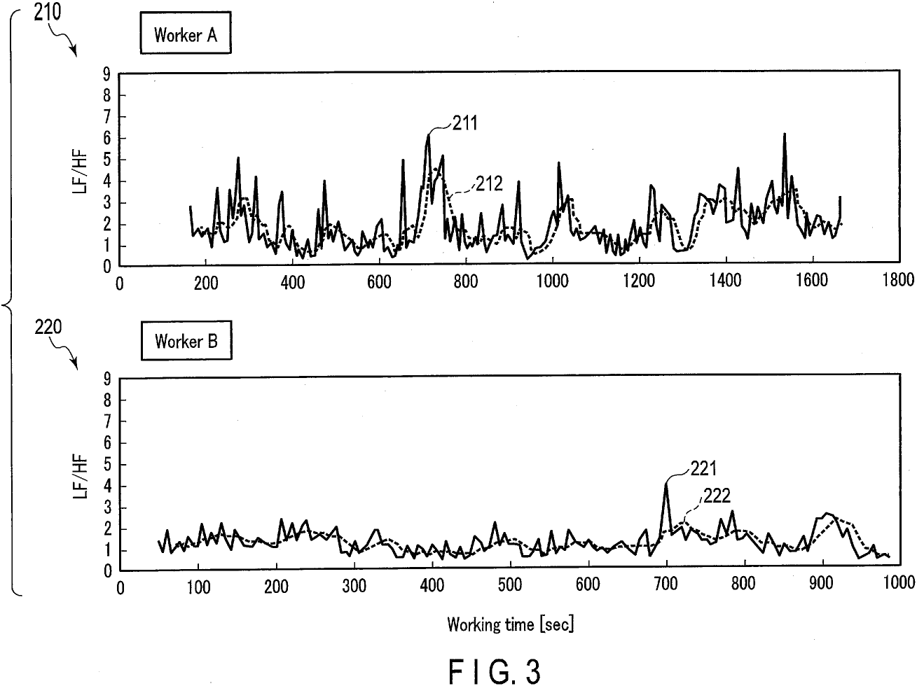

The time-series data about the change in NN interval requires a predetermined period such as at least one minute in order to perform frequency analysis. This analyzing method calculates one LF/HF ratio for this predetermined period. The LF/HF ratio can be calculated for each of one-minute periods not overlapping each other, and can also be calculated for each of one-minute periods that overlap each other by shifting for a predetermined number of heartbeats or a predetermined number of seconds. That is, the time-series data about the LF/HF ratio is calculated by calculating the LF/HF ratio from the time-series data about the change in NN interval at an arbitrary timing (see FIG. 3). Note that it is also possible to calculate the moving average for every predetermined period or every predetermined time length with respect to the calculated time-series data about the LF/HF ratio.

In step S103, the proficiency determination apparatus 1 analyzes the LF/HF ratio or the moving average by the physiological index analyzing unit 103. For example, the proficiency determination apparatus 1 calculates the frequency of a local maximum of the LF/HF ratio by analyzing the time-series data about the LF/HF ratio. A practical method of calculating the frequency of a local maximum of the LF/HF ratio will be described below in steps S104 to S107.

In step S104, the proficiency determination apparatus 1 determines whether the LF/HF ratio at an arbitrary time (time = tₓ) is larger than the LF/HF ratio at a time (time = tₓ₋₁) immediately before the arbitrary time by a predetermined value or more by using the physiological index analyzing unit 103. If this proposition is true (Yes in step S104), the process advances to step S105. If the proposition is false (No in step S104), the process advances to step S106.

The arbitrary time is set for each timing at which the LF/HF ratio is calculated. For example, assume that time-series data is obtained by calculating the LF/HF ratio at each of a plurality of temporally successive times (t0, t1, t2, and t3). When initially executing step S104, the proficiency determination apparatus 1 sets the first time to as an arbitrary time. However, step S104 is not executed because there is no time immediately before the time t0. Therefore, the proficiency determination apparatus 1 sets the time t1 immediately after the time to as an arbitrary time. The proficiency determination apparatus 1 determines whether the LF/HF ratio at the time t1 is larger than the LF/HF ratio at the time to by a predetermined value or more. Step S104 is repetitively executed until the proficiency determination apparatus 1 sets the final time t3 as an arbitrary time.

In step S105, the proficiency determination apparatus 1 sets a frequency N of the physiological index to N + 1 (N = N + 1) by using the physiological index analyzing unit 103. In other words, the frequency of the local maximum for the LF/HF ratio increases by 1 whenever step S105 is executed.

In step S106, the proficiency determination apparatus 1 sets an arbitrary time to a time immediately after the arbitrary time (time = tₓ₊₁) by using the physiological index analyzing unit 103. In other words, an arbitrary time is set to a time immediately after the arbitrary time whenever step S106 is executed.

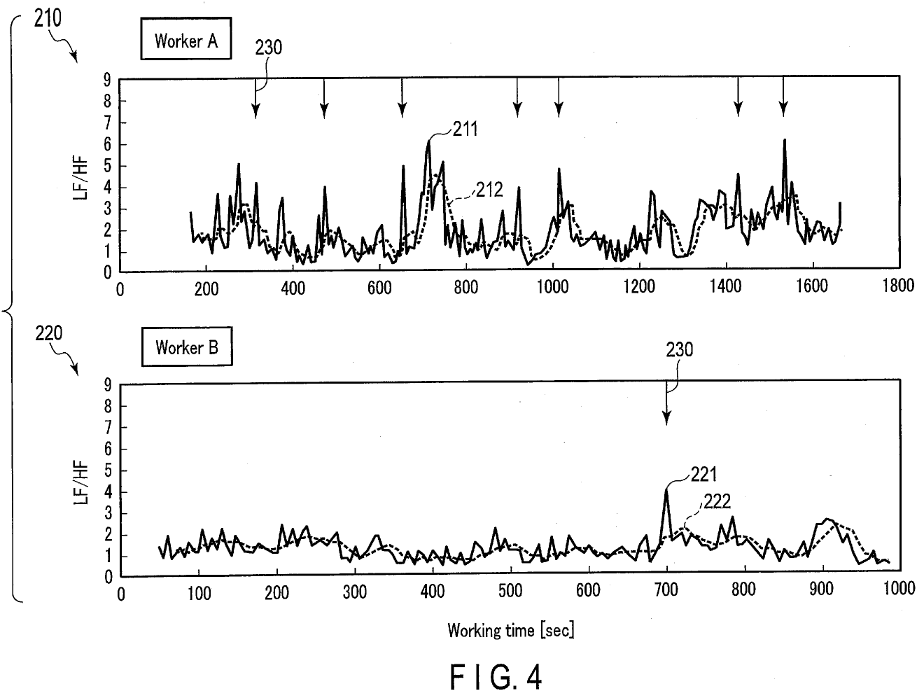

In step S107, the proficiency determination apparatus 1 determines whether an arbitrary time is set to the final time (time = t_{end}?) by using the physiological index analyzing unit 103. If this proposition is true (Yes in step S107), the process advances to step S108. If the LF/HF ratio at the final time is higher than the LF/HF ratio at a time immediately before the final time by a predetermined value or more, the proficiency determination apparatus 1 can of course increase the frequency of the local maximum by 1. Consequently, the frequency of the local maximum in the time-series data about the LF/HF ratio is calculated (see FIG. 4). If the proposition is false (No in step S107), the process returns to step S104.

In step S108, the proficiency determination apparatus 1 determines whether the frequency N of the physiological index is “3” or more (N ≥ 3?) by using the proficiency determining unit 104. More specifically, whether the calculated frequency of the local maximum for the LF/HF ratio is “3” or more is determined. If this proposition is true (Yes in step S108), the process advances to step S109. If the proposition is false (No in step S108), the process advances to step S110. The threshold of the frequency N of the physiological index is of course not limited to “3” and can take an arbitrary value of “1” or more.

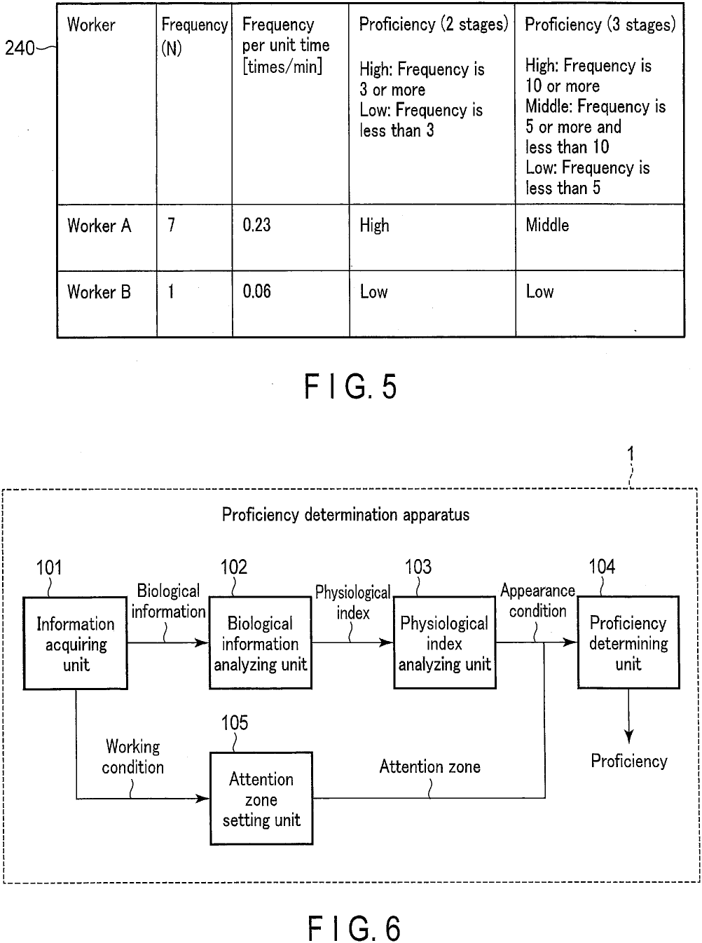

In step S109, the proficiency determination apparatus 1 determines that the proficiency of the worker is high by using the proficiency determining unit 104, and outputs this determination result (proficiency: high) (see FIG. 5). After step S109, the proficiency determination apparatus 1 terminates the series of processes.

In step S110, the proficiency determination apparatus 1 determines that the proficiency of the worker is low by using the proficiency determining unit 104, and outputs this determination result (proficiency: low) (see FIG. 5). After step S110, the proficiency determination apparatus 1 terminates the series of processes.

The operation example of the proficiency determination apparatus 1 according to the first embodiment has been explained above. As described above, the proficiency determination apparatus 1 determines that the proficiency is high when the frequency N of the physiological index is equal to or larger than the threshold. Generally, a worker having a high proficiency pays attention to various surrounding things, so the worker presumably pays attention not only to the peripheral environment but also to the quality of a product during a work. Accordingly, when the frequency N of the physiological index indicating the mental stress on a worker during a work is high, the worker is assumed to pay attention to the quality of a product as well. Based on this assumption, the proficiency determination apparatus 1 determines that a worker has a high proficiency for the quality evaluation ability when the frequency N of the physiological index is equal to or larger than the threshold.

FIG. 3 is a view showing an example of the time-series data about the LF/HF ratio according to the first embodiment. Referring to FIG. 3, a graph 210 indicating the time-series data about the LF/HF ratio of “worker A” and a graph 220 indicating the time-series data about the LF/HF ratio of “worker B” are so arranged that they can be compared. In the graphs 210 and 220, the ordinate indicates the value of the LF/HF ratio within the range of “0 to 9”, and the abscissa indicates the working time in units of seconds. The range of the working time of the graph 210 is “0 to 1,800 sec”, and the range of the working time of the graph 220 is “0 to 1,000 sec”. The graph 210 includes solid-line time-series data 211 indicating a change in measurement value of the LF/HF ratio with time of “worker A”, and dotted-line time-series data 212 indicating a change in moving average with time thereof. The graph 220 includes solid-line time-series data 221 indicating a change in measurement value of the LF/HF ratio with time of “worker B”, and dotted-line time-series data 222 indicating a change in moving average with time thereof.

As can be understood from the graphs 210 and 220, the measurement value of the LF/HF ratio of worker A frequently and abruptly changes compared to the measurement value for worker B. In other words, a strong mental stress is frequently applied on worker A compared to worker B. By analyzing the graphs 210 and 220, the proficiency determination apparatus 1 calculates, for each graph, the timing and frequency at which a peak (local maximum) indicating an abrupt transient rise of the measurement value of the LF/HF ratio appears.

FIG. 4 is a view showing an example of the frequency of a local maximum in the time-series data about the LF/HF ratio according to the first embodiment. Referring to FIG. 4, when the measurement value of the LF/HF ratio at a specific timing is larger than the measurement value of the LF/HF ratio at a timing before the specific timing by a predetermined value or more, the former measurement value is calculated as a local maximum. In FIG. 4, downward arrows 230 indicate the timings of local maximums calculated for the graphs 210 and 220. Each downward arrow 230 indicates a local maximum of the time-series data 211 or 221. More specifically, seven local maximums are calculated in the graph 210. In other words, an instantaneous mental stress is applied on worker A seven times during the working time. On the other hand, one local maximum is calculated in the graph 220. In other words, an instantaneous mental stress is applied on worker B once during the working time. The proficiency determination apparatus 1 processes the number of calculated local maximums as the frequency N of the physiological index.

FIG. 5 is a view showing an example of the correspondence between the frequency of a local maximum and the proficiency in the time-series data about the LF/HF ratio according to the first embodiment. FIG. 5 shows a table 240 indicating the correspondence between the frequency N of the physiological index and the proficiency. The table 240 includes items “worker”, “frequency (N) “, “frequency per unit time”, “proficiency (2 stages)", and ”frequency (3 stages)”. According to the above-described calculation results, the frequency N for worker A is “7”, and the frequency per unit time is calculated to be “0.23” (equation: 7 [times]/(1800/60) [min] ) . Similarly, the frequency N for worker B is “1”, and the frequency per unit time is calculated to be “0.06” (equation: 1 time]/(1000/60) [min]).

The proficiency determination apparatus 1 determines the proficiency of each worker based on the frequency N of the physiological index calculated for each worker. When determining the proficiency by two stages, the proficiency determination apparatus 1 determines that the proficiency is “proficiency: high” when the frequency is “3” or more, and “proficiency: low” when the frequency is less than “3”. According to this criterion, the frequency “7” of worker A is “3” or more, so it is determined that the proficiency of worker A is high. On the other hand, the frequency “1” of worker B is less than “3”, so it is determined that the proficiency of worker B is low. The proficiency determination apparatus 1 thus determines that worker A is an “expert” and worker B is a “beginner”.

When determining the proficiency by three stages, the proficiency determination apparatus 1 determines that the proficiency is “proficiency: high” when the frequency is “10” or more, “proficiency: middle” when the frequency is “5” or more and less than “10”, and “proficiency: low” when the frequency is less than “5”. According to this criterion, the frequency “7” of worker A is “5” or more and less than “10”, so it is determined that the proficiency of worker A is middle. On the other hand, the frequency “1” of worker B is less than “5”, so it is determined that the proficiency of worker B is low. The proficiency determination apparatus 1 thus determines that worker A is an “intermediate” and worker B is a “beginner”.

As described above, the proficiency determination apparatus 1 can determine the proficiency by two stages based on whether the frequency of the physiological index is equal to or larger than one threshold, and can also determine the proficiency by three or more stages based on a plurality of thresholds. The proficiency determination apparatus 1 can of course determine the proficiency by similarly classifying the proficiency, based on the frequency per unit time. In this embodiment, it is assumed that the proficiency is a value indicating a plurality of categories (high, middle, and low). However, the proficiency can also be a value indicated by a ratio such as “0 to 100%”.

The proficiency determination apparatus 1 according to the first embodiment has been explained above. The proficiency determination apparatus 1 determines the proficiency of a worker based on the appearance condition of the physiological index indicating the mental stress during a work. Accordingly, the proficiency determination apparatus 1 can measure the quality evaluation ability of the worker, which cannot be measured from objective information such as the working time, the action, or the posture. In addition, even when many products manufactured by a worker has high quality with no problem, the proficiency determination apparatus 1 can determine whether the worker is performing works on the products with confidence. Therefore, the proficiency determination apparatus 1 can qualitatively and quantitatively evaluate the occurrence probability of a potential work error of the worker, which cannot be evaluated from the test result of the quality of a product as an end product. That is, the proficiency determination apparatus 1 can determine the proficiency for the quality evaluation ability of a worker.

### Second Embodiment

FIG. 6 is a block diagram showing a configuration example of a proficiency determination apparatus 1 according to the second embodiment. The proficiency determination apparatus 1 according to the second embodiment further includes an attention zone setting unit 105 in addition to the components included in the proficiency determination apparatus 1 according to the first embodiment. The attention zone setting unit 105 is an example of a setting unit. An information acquiring unit 101 according to the second embodiment is an example of a second acquiring unit.

The information acquiring unit 101 acquires the working condition of a worker. More specifically, the information acquiring unit 101 acquires third time-series data about the working condition of a worker in a predetermined period. The working condition is information indicating a working zone in which a worker is performing a work during a predetermined period, a waiting zone in which the worker is waiting, and a working step zone in which the worker is executing a specific working step in the working zone. The specific working step includes assembling, processing, making, and quality check of a product. That is, the working condition is information indicating the type of work which a worker is executing at a predetermined timing in the working time. More specifically, the working condition is information indicating whether a worker is performing a work, waiting, or executing a specific working step. The working condition can be acquired from various kinds of information (e.g., an acceleration, an angular velocity, and an image) indicating the action of a worker and the movement of a product existing around the worker. It is also possible to acquire the working condition from a declaration by a worker or a declaration by another worker existing around the worker. Subsequently, the information acquiring unit 101 outputs the acquired third time-series data about the working condition to the attention zone setting unit 105.

In this embodiment, it is assumed that the working time is split into a working zone and a waiting zone. A first method of distinguishing between the working zone and the waiting zone is a method of using the acceleration or the angular velocity obtained from a motion sensor installed on a worker. If there is a tendency that the absolute value of the acceleration or the angular velocity in the waiting zone is larger than that in the working zone, it is possible to distinguish between the working zone and the waiting zone based on the absolute value. Alternatively, if a specific work has a unique waveform of the acceleration or the angular velocity, it is possible to distinguish between the working zone and the waiting zone by analyzing the waveform by a machine learning model.

A second method is a method of using an image obtained by taking an image of the action of a worker. According to this method, the image is first analyzed by a human detection technique or a skeleton estimation technique, thereby tracking the position or action of the worker. Subsequently, the tracked position or action is analyzed, thereby specifying a period in which the worker takes a specific action or posture as a working zone, and a period in which the worker does not take the specific action or posture as a waiting zone. Alternatively, a period in which the worker exists in a workplace is specified as a working zone, and a period in which the worker exists in a waiting place is specified as a waiting zone.

A third method is a method of using the acceleration or the angular velocity acquired from a motion sensor installed on a product. According to this method, it is possible to distinguish between the working zone and the waiting zone by determining whether a manufacturing line is in operation based on the acceleration or the angular velocity of the product. Another means such as a laser that specifies the product position can also be used. It is of course also possible to calculate and acquire the working condition by performing the above-described first, second, or third method by the proficiency determination apparatus 1.

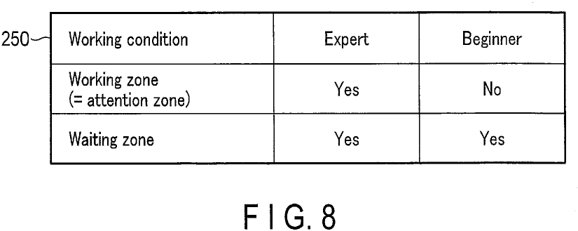

The attention zone setting unit 105 sets an attention zone. More specifically, the attention zone setting unit 105 sets an attention zone for the third time-series data about the working condition of a worker. For example, the attention zone setting unit 105 sets a working zone or a working step zone as the attention zone. Generally, a worker pays attention when to start a next work in a waiting zone, so the mental stress presumably rises for both an expert and a beginner due to a feeling of tension. Therefore, it is assumed that in a working zone different from a waiting zone, a difference is produced in the appearance characteristic of the physiological index indicating the mental stress between an expert and a beginner (see FIG. 8). Based on this assumption, the attention zone setting unit 105 sets the working zone as an attention zone. Subsequently, the attention zone setting unit 105 outputs information about the set attention zone to a proficiency determining unit 104.

The proficiency determining unit 104 determines the proficiency of the worker based on the appearance condition of the physiological index and the attention zone. More specifically, the proficiency determining unit 104 determines the proficiency of the worker based on the appearance condition of the physiological index in the attention zone. The proficiency determination method is the same as that of the first embodiment. If a plurality of set attention zones exist, the proficiency determining unit 104 can totalize the frequencies of the physiological indices calculated in the individual attention zones. Furthermore, the proficiency determining unit 104 can calculate the frequency per unit time for the totalized physiological index frequency. Subsequently, the proficiency determining unit 104 outputs the determined proficiency of the worker to the inside or the outside of the proficiency determination apparatus 1.

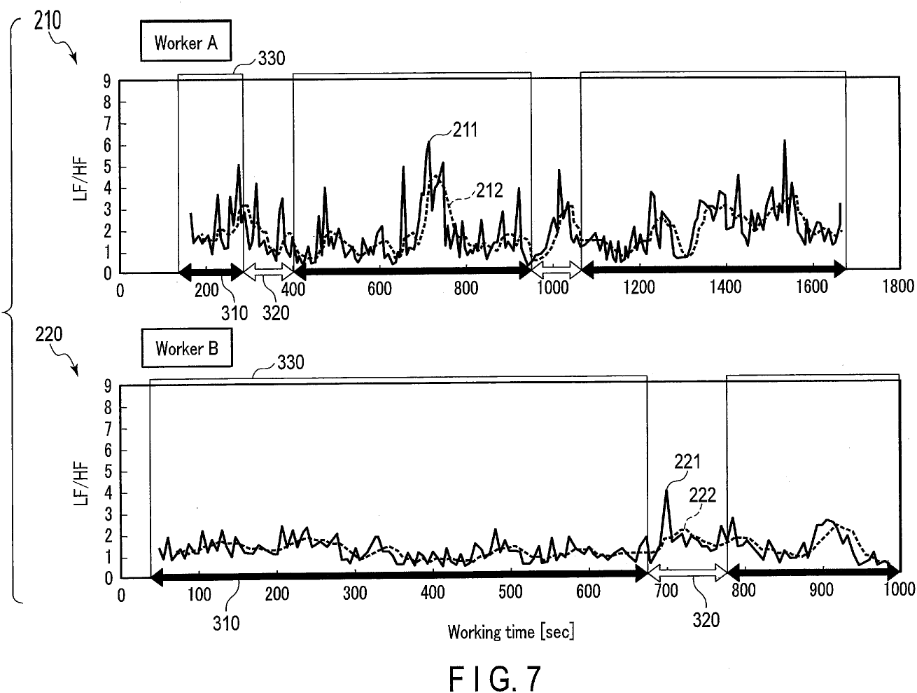

FIG. 7 is a view showing an example of the working condition of a worker corresponding to time-series data about an LF/HF ratio according to the second embodiment. Referring to FIG. 7, the working zone and the waiting zone indicating the working condition and the set attention zone are superposed on each other in graphs 210 and 220. A black double-headed arrow 310 indicates the working zone, a white double-headed arrow 320 indicates the waiting zone, and a rectangular frame 330 indicates the attention zone. The graph 210 shows three working zones and two waiting zones existing between the three working zones. The graph 220 shows two working zones and one waiting zone existing between the two working zones.

As can be understood from the graphs 210 and 220, a measurement value of the LF/HF ratio of worker A frequently and abruptly changes regardless of the working zone and the waiting zone. On the other hand, a measurement value of the LF/HF ratio of worker B abruptly changes particularly in the waiting zone. The proficiency determination apparatus 1 analyzes the graphs 210 and 220 while setting the working zone as the attention zone, thereby calculating the timing and the frequency at which a peak (local maximum) indicating an abrupt transient rise of a measurement value of the LF/HF ratio appears in the attention zone of each graph.

FIG. 8 is a view showing the appearance characteristic of the physiological index corresponding to the working condition of a worker according to the second embodiment. FIG. 8 shows a table 250 indicating the correspondence between the working condition and the appearance characteristic of the physiological index of an expert or a beginner. As the table 250 shows, it is assumed that in the working zone, a physiological index appears for the expert but does not appear for the beginner. On the other hand, it is assumed that in the waiting zone, the physiological index appears for both the expert and the beginner. In other words, it is assumed that in the working zone, a difference is observed in the appearance characteristic of the physiological index between the expert and the beginner. Based on this assumption, the proficiency determination apparatus 1 sets the working zone as the attention zone.

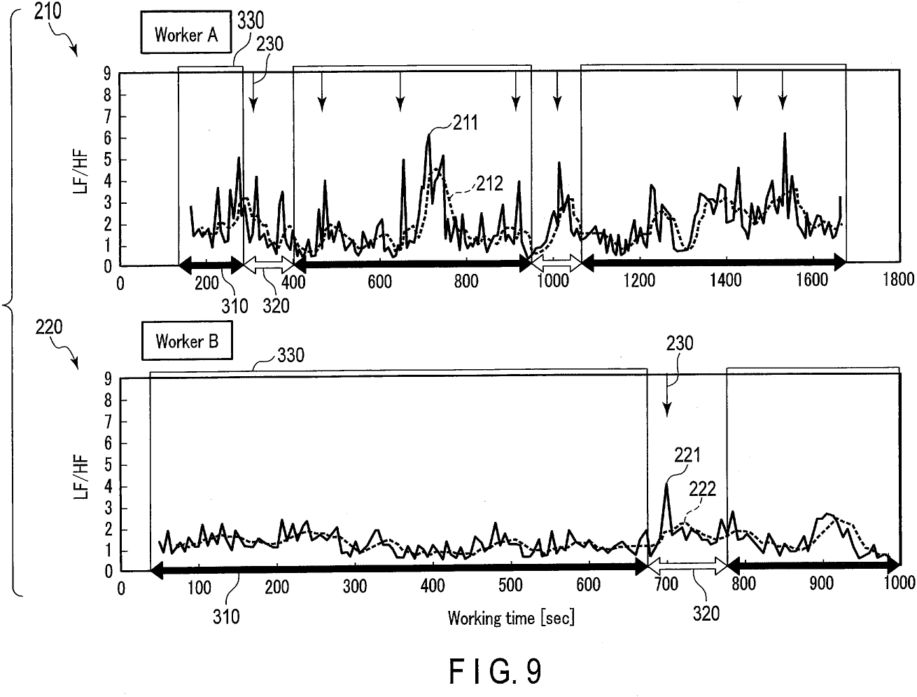

FIG. 9 is a view showing an example of the frequency of a local maximum in the time-series data about the LF/HF ratio according to the second embodiment. Referring to FIG. 9, a downward arrow 230 indicates the timing of a local maximum calculated for the graphs 210 and 220. More specifically, in the graph 210, a total of five local maximums are calculated in three working zones, and a total of two local maximums are calculated in two waiting zones. In other words, an instantaneous mental stress is applied on worker A five times in the working zone and twice in the waiting zone. On the other hand, in the graph 220, no local maximum is calculated in two working zones, and one local maximum is calculated in one waiting zone. In other words, no instantaneous mental stress is applied on worker B in the working zone, and an instantaneous mental stress is applied on worker B once in the waiting zone. The proficiency determination apparatus 1 processes the number of local maximums calculated in the working zone as a frequency N of the physiological index.

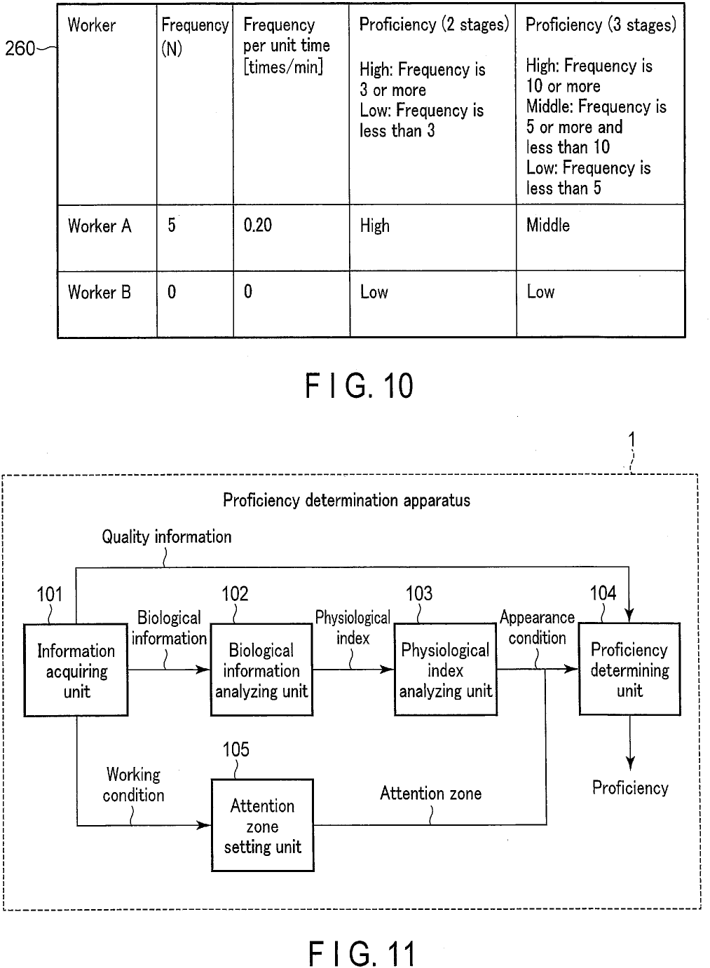

FIG. 10 is a graph showing an example of the correspondence between the frequency of a local maximum and the proficiency in the time-series data about the LF/HF ratio according to the second embodiment. FIG. 10 shows a table 260 indicating the correspondence between the frequency N of the physiological index and the proficiency. The table 260 shows items “worker”, “frequency (N)”, “frequency per unit time”, “proficiency (2 stages)”, and “proficiency (3 stages)”. According to the above-described calculation results, the frequency N is “5” and the frequency per unit time is “0.20” for worker A. On the other hand, the frequency N is “0” and the frequency per unit time is “0” for worker B.

The proficiency determination apparatus 1 determines the proficiency of each worker based on the same criterion as that of the first embodiment. When determining the proficiency by two stages, it is determined that the proficiency of worker A is high because the frequency “5” of worker A is “3” or more. On the other hand, it is determined that the proficiency of worker B is low because the frequency “0” of worker B is less than “3”. Thus, the proficiency determination apparatus 1 determines that worker A is an “expert” and worker B is a “beginner”.

When determining the proficiency by three stages, it is determined that the proficiency of worker A is middle because the frequency “5” of worker A is “5” or more and less than “10”. On the other hand, it is determined that the proficiency of worker B is low because the frequency “0” of worker B is less than 5”. Thus, the proficiency determination apparatus 1 determines that worker A is an “intermediate” and worker B is a “beginner”.

The proficiency determination apparatus 1 according to the second embodiment has been explained above. The proficiency determination apparatus 1 determines the proficiency of a worker based on the appearance condition of the physiological index in the attention zone. Therefore, the proficiency determination apparatus 1 can determine the quality evaluation ability of the worker based on the appearance condition of the physiological index in a specific time period of the working time of the worker. Also, the proficiency determination apparatus 1 can measure the quality evaluation ability of a worker by paying attention to a time period (a working step zone) during which the worker is engaged in a specific work (e.g., assembling, processing, making, or quality check).

### Third Embodiment

FIG. 11 is a block diagram showing a configuration example of a proficiency determination apparatus 1 according to the third embodiment. The proficiency determination apparatus 1 according to the third embodiment includes the same components as those of the proficiency determination apparatus 1 according to the second embodiment. An information acquiring unit 101 according to the third embodiment is an example of a third acquiring unit.

The information acquiring unit 101 acquires quality information of a product. More specifically, the information acquiring unit 101 acquires quality information indicating the quality of a product having undergone a work performed by a worker. For example, the quality information is information indicating the quality of a product is high or low. In this embodiment, it is assumed that the quality information is evaluated by two stages. In this case, the quality information is expressed as, e.g., good product/defective product, proper/improper, good/defective, or O/X. The quality information can also be evaluated by three or more stages. In this case, the quality information is expressed as, e.g., good/average/defective or O / 0/x. The quality information can of course be a value indicating a plurality of categories or a value indicating a ratio such as “0 to 100%”. The quality information can also be acquired from, e.g., the result of visual judgement on a product by a human, the result of an automatic calculation on an image of a product by a machine learning model, or the result of an objective test on the quality of a product. Subsequently, the information acquiring unit 101 outputs the acquired quality information to a proficiency determining unit 104.

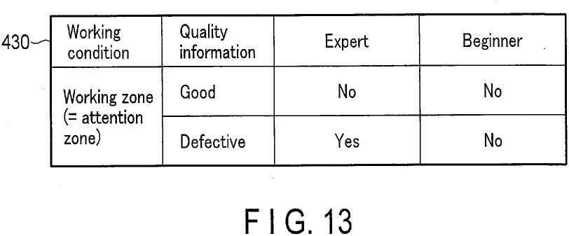

An attention zone setting unit 105 sets a checking zone in which a worker checks the quality of a product in a working zone as an attention zone. In this embodiment, it is assumed that the working zone includes a period (checking zone) in which a worker checks the quality of a product after a work for assembling, processing, or making a product. It is also assumed that the quality information is related to the checking zone. Generally, it is considered that an expert can sufficiently recognize the quality of a product but a beginner cannot sufficiently recognize the quality of a product. It is also considered that when an expert recognizes that the quality of a product is high, he or she does not recognize that the product needs correction, so the mental stress does not rise. On the other hand, it is believed that when an expert recognizes that a product is defective, he or she recognizes that the product needs correction, so the mental stress rises. By contrast, it is believed that a beginner cannot sufficiently recognize the quality of a product, so the mental stress does not rise regardless of the quality of the product. Accordingly, it is assumed that when a product is defective, a difference is probably produced in the appearance characteristic of a physiological index indicating the mental stress between an expert and a beginner (see FIG. 13). Based on this assumption, the attention zone setting unit 105 sets the checking zone as an attention zone. Subsequently, the attention zone setting unit 105 outputs information about the set attention zone to the proficiency determining unit 104.

The proficiency determining unit 104 determines the proficiency of the worker based on the appearance condition of the physiological index in the attention zone and the quality information. More specifically, the proficiency determining unit 104 determines the proficiency of the worker based on the appearance condition of the physiological index in the attention zone when the product is defective. In this embodiment, the proficiency determining unit 104 determines whether the worker recognizes that the product is defective, based on whether a local maximum of an LF/HF ratio appears in the checking zone. Subsequently, the proficiency determining unit 104 outputs the determined proficiency of the worker to the inside or the outside of the proficiency determination apparatus 1.

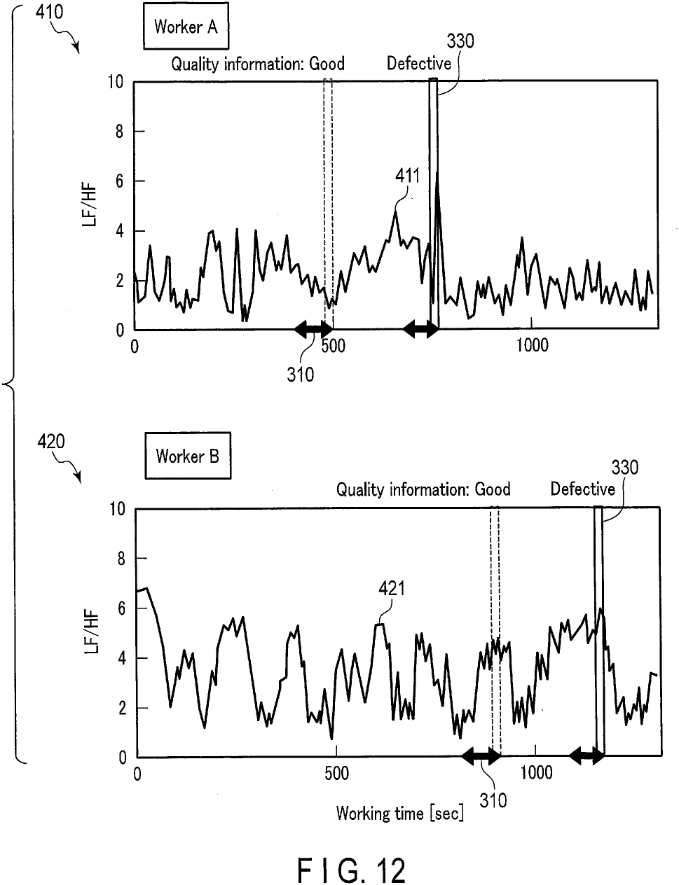

FIG. 12 is a view showing an example of the correspondence between the working zone and the quality information of a product in the time-series data about the LF/HF ratio according to the third embodiment. Referring to FIG. 12, a graph 410 indicating the time-series data about the LF/HF ratio of “worker A” and a graph 420 indicating the time-series data about the LF/HF ratio of “worker B” are arranged so that they can be compared. In the graphs 410 and 420, the ordinate indicates the value of the LF/HF ratio by the range of “0 to 10”, and the abscissa indicates the working time in units of seconds. The graph 410 contains solid-line time-series data 411 indicating a change in measurement value of the LF/HF ratio with time of “worker A”. The graph 420 contains solid-line time-series data 421 indicating a change in measurement value of the LF/HF ratio with time of “worker B”.

In addition, the working zone indicating the working condition and the set attention zone are superposed on each other in the graphs 410 and 420. A double-headed arrow 310 indicates the working zone, and a rectangular frame 330 indicates the attention zone. In this case, it is assumed that the attention zone and the checking zone have the same range. Each of the graphs 410 and 420 shows two working zones and two checking zones positioned near the end points of the two working zones. More specifically, in each of the graphs 410 and 420, quality information for the first checking zone is “good”, and that for the second checking zone is “defective”. In the same manner as in the first and second embodiments, the proficiency determination apparatus 1 analyzes the graphs 410 and 420, thereby calculating, for each graph, the timing and the frequency at which a peak (local maximum) indicating an abrupt transient change of the measurement value of the LF/HF ratio appears.

FIG. 13 is a view showing the appearance characteristic of the physiological index corresponding to the quality information of a product according to the third embodiment. FIG. 13 shows a table 430 indicating the correspondence between the quality information of the appearance characteristic of the physiological index of an expert or a beginner. In the table 430, attention will be focused on the quality information in the checking zone. As shown in the table 430, it is assumed that when the quality information is “good”, no physiological index appears for both the expert and the beginner. On the other hand, it is assumed that when the quality information is “defective”, a physiological index appears for the expert but does not appear for the beginner. Based on these assumptions, the proficiency determination apparatus 1 sets the checking zone when the quality information is defective as an attention zone.

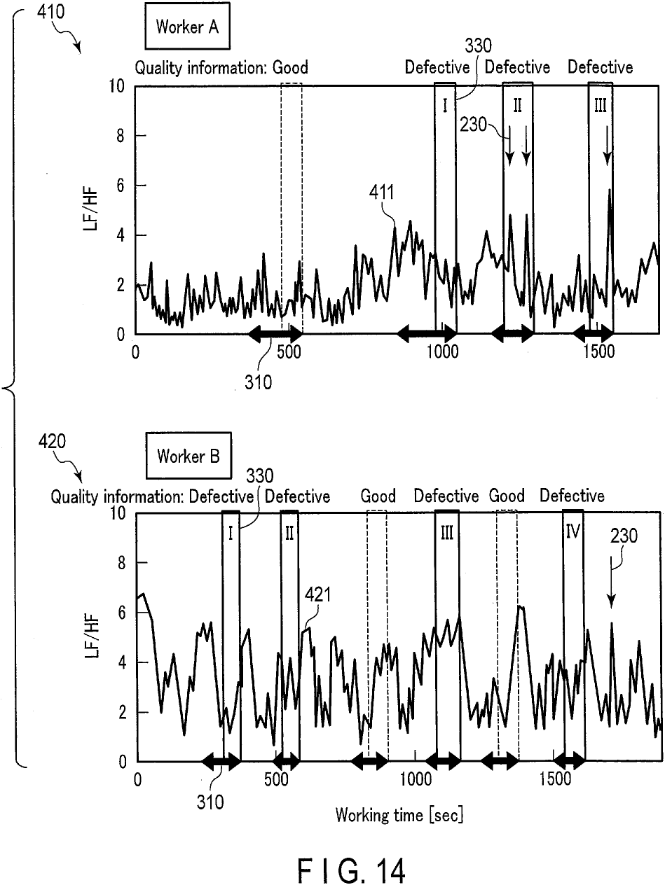

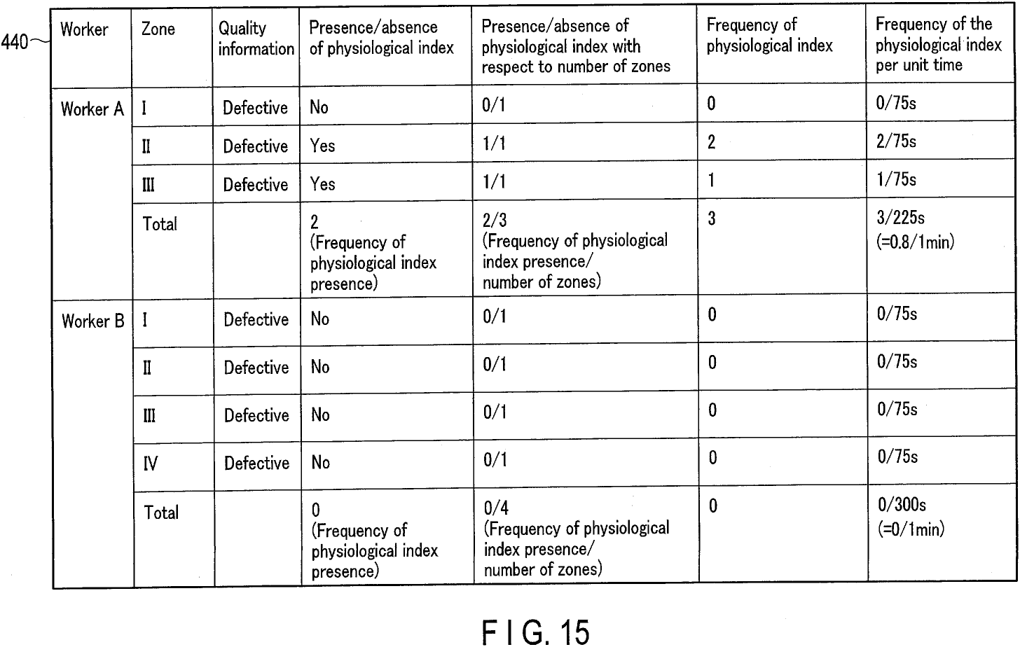

FIGS. 14 and 15 are views showing analytical examples of the frequency of a local maximum in the time-series data about the LF/HF ratio according to the third embodiment. In FIG. 14, each of downward arrows 230 indicates the timing of the local maximum of the physiological index calculated for the graphs 410 and 420. The downward arrow 230 indicates the local maximum of the time-series data 411 and 421. More specifically, a total of three local maximums are calculated in three attention zones (I, II, and III) of the graph 410. In other words, an instantaneous mental stress is applied on worker A three times in total in the checking zones. On the other hand, no local maximum is calculated in any of four attention zones (I, II, III, and IV) of the graph 420. In other words, no instantaneous mental stress is applied on worker B in any checking zone. In the same manner as in the first and second embodiments, the proficiency determination apparatus 1 processes the calculated number of local maximums as a frequency N of the physiological index.

FIG. 15 shows a table 440 indicating the analytical results of the graphs 410 and 420. As shown in the table 440, when the quality information is “defective” in a plurality of zones, the proficiency determination apparatus 1 can totalize the presence/absence or the frequency of the physiological index in each zone. For example, when the quality information is “defective” in three attention zones as shown in the graph 410, the proficiency determination apparatus 1 can calculate the items (the presence/absence of the physiological index, the presence/absence of the physiological index with respect to the number of zones, the frequency of the physiological index, and the frequency of the physiological index per unit time) for each attention zone. It is of course also possible to totalize the presence/absence or the frequency of the physiological index in each zone for the graph 420 as well by the same method. In the graph 410, the physiological index appears in two attention zones in which the quality information is “defective”. On the other hand, no physiological index appears in any attention zone in the graph 420, so all total values of the items described above are “0”. Thus, the proficiency determination apparatus 1 can determine that worker A is an “expert” or an “intermediate” and worker B is a “beginner” from the graphs 410 and 420 in FIG. 14 as well.

The proficiency determination apparatus 1 according to the third embodiment has been explained above. The proficiency determination apparatus 1 determines the proficiency of a worker based on the appearance condition of the physiological index and the quality information in the attention zone. Accordingly, the proficiency determination apparatus 1 can measure the quality evaluation ability of the worker by focusing attention on the quality information of a product. More specifically, the proficiency determination apparatus 1 can determine the proficiency in accordance with the appearance condition of the physiological index in the checking zone when the product is defective.

### Fourth Embodiment

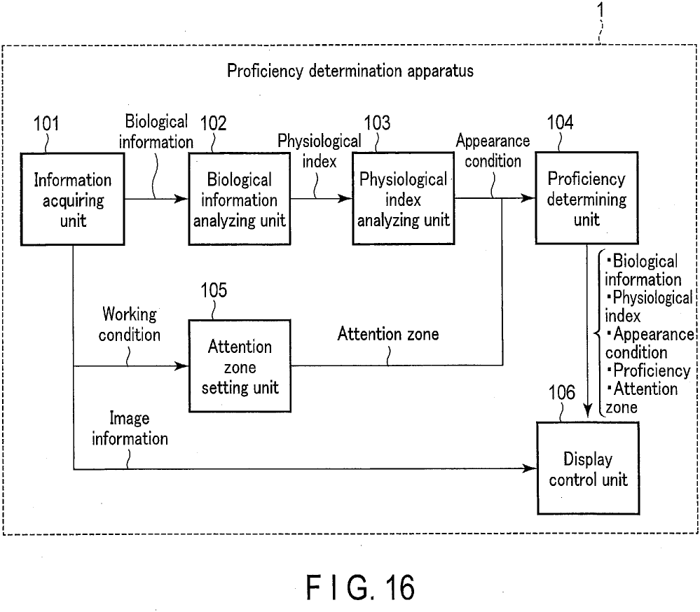

FIG. 16 is a block diagram showing a configuration example of a proficiency determination apparatus 1 according to the fourth embodiment. The proficiency determination apparatus 1 according to the fourth embodiment further includes a display control unit 106 in addition to the components included in the proficiency determination apparatus 1 according to the second embodiment. The display control unit 106 is an example of a display controller. An information acquiring unit 101 according to the fourth embodiment is an example of a fourth acquiring unit.

The information acquiring unit 101 acquires image information of a worker. More specifically, the information acquiring unit 101 acquires image information indicating the action of a worker in a predetermined period. This image information need only be data about the same period as that of data about biological information of the worker. The image information can also be acquired from a camera installed in a position where the camera can image the worker. Subsequently, the information acquiring unit 101 outputs the acquired image information to the display control unit 106.

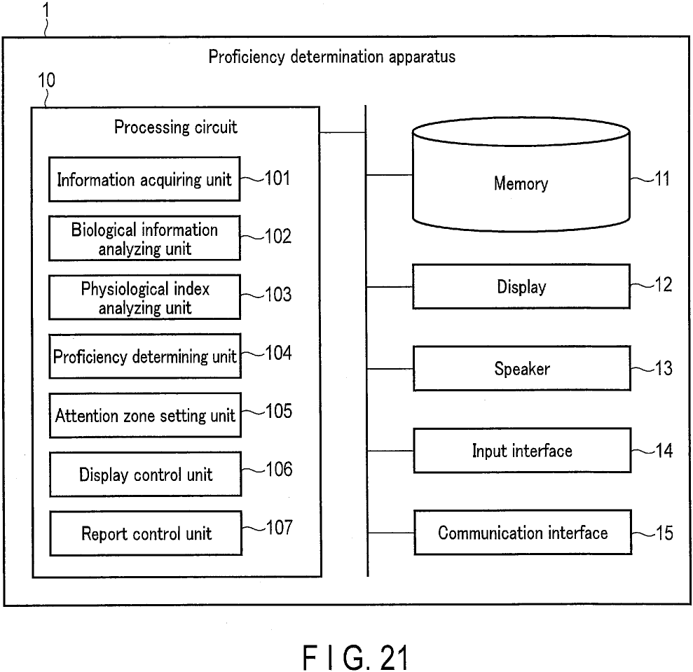

The display control unit 106 displays various kinds of information about the worker. For example, the display control unit 106 displays display data obtained by relating at least one of first time-series data, second time-series data, the appearance condition of the physiological index, and the proficiency of the worker to the image information. The display control unit 106 can of course display an attention zone set by an attention zone setting unit 105 by relating the attention zone to the image information. Also, in relation to the display data of a current work of a worker, the display control unit 106 can display the display data of a past work of the current worker or another worker. As another mode, in relation to the display data of a current work of a worker, the display control unit 106 can display the display data of a past work of another worker having a proficiency higher than that of the current worker. That is, the display control unit 106 can simultaneously display two kinds of display data such that the display data can be compared to each other. The display control unit 106 displays the display data on, e.g., a display 12 (see FIG. 21).

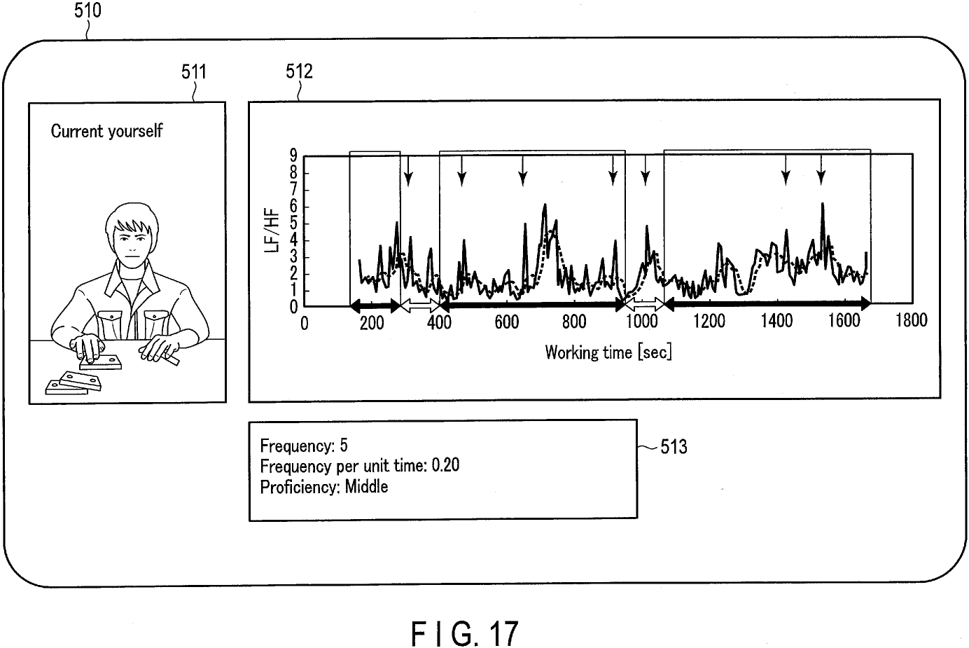

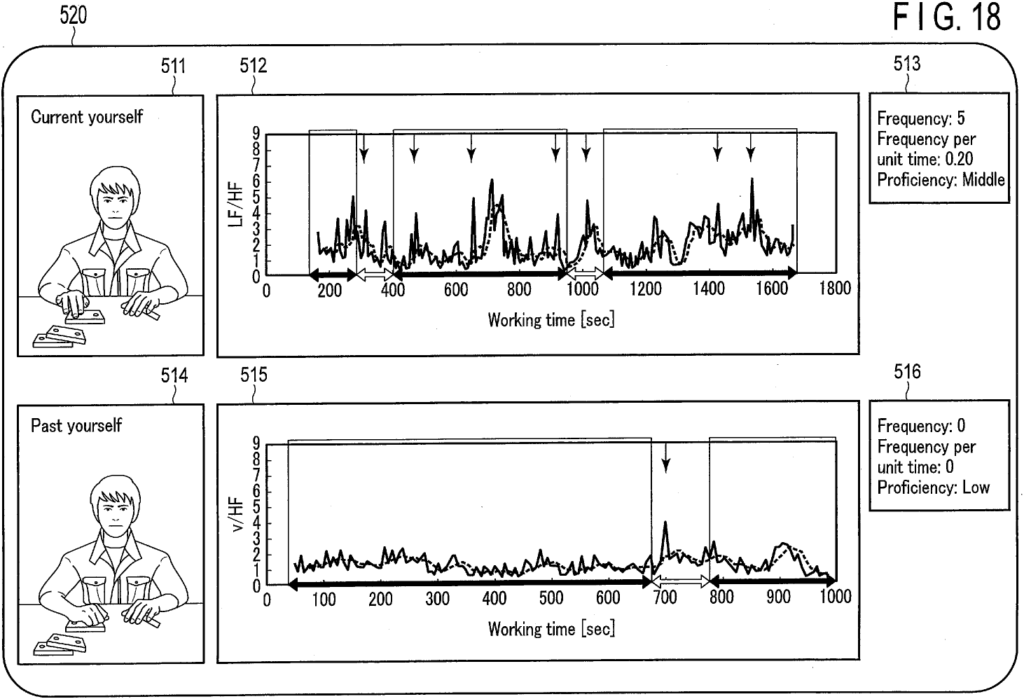

FIGS. 17 and 18 are views showing examples of display data 510 and 520 according to the fourth embodiment. The display data 510 shown in FIG. 17 includes an area 511 for displaying image information of a worker in a current work, an area 512 for displaying the physiological index of the current worker, the appearance condition of the physiological index, and an attention zone, and an area 513 for displaying the proficiency of the current worker. More specifically, the area 511 displays an image indicating the action of a worker in a current work. The time range of this image is equivalent to the range of working time “0 to 1800 sec” of a graph displayed in the area 512. On the other hand, the area 512 displays a graph similar to the graph 210 (see FIG. 9). Note that the current playback position of the image played back in the area 511 can also be indicated by a straight line or the like on the graph in the area 512. The area 513 displays the contents of the table 260 (see FIG. 10).

The display data 520 shown in FIG. 18 includes an area 514 for displaying image information of a worker in a past work, an area 515 for displaying the appearance condition of the physiological index and an attention zone, and an area 516 for displaying the proficiency of the past worker, in addition to the areas 511, 512, and 513. That is, various kinds of information on a current work and a past work of the same worker are simultaneously displayed. More specifically, the area 514 displays an image showing the action of a worker in a past work. The time range of this image is equivalent to the range of working time “0 to 1000 sec” of a graph displayed in the area 515. The area 515 displays a graph similar to the graph 220 (see FIG. 9). Note that the current playback position of the image played back in the area 514 can be indicated by a straight line or the like on the graph of the area 515. The area 516 displays contents contained in the table 260 (see FIG. 10). Note that the information displayed in the areas 514, 515, and 516 can be either past information of a current worker or past information of a different worker. For example, the proficiency determination apparatus 1 can select a worker having a proficiency higher than that of a specific worker, and display information about a past work executed by the selected worker in the areas 514, 515, and 516. Consequently, the proficiency determination apparatus 1 allows a worker to compare an image obtained during a work and the appearance condition of the physiological index with those of another worker having a high proficiency. That is, the proficiency determination apparatus 1 can help a worker make efforts to spontaneously improve the technique based on comparison with other workers.

The proficiency determination apparatus 1 according to the fourth embodiment has been explained above. The proficiency determination apparatus 1 displays display data obtained by relating at least one of the biological information, the physiological index, the appearance condition of the physiological index, the proficiency, and the attention zone to image information. Consequently, the proficiency determination apparatus 1 allows a worker to check his or her proficiency and action in real time during a work or after the work. For example, a worker can check whether his or her proficiency has improved in a current work by checking the appearance condition of the physiological index and an image obtained when he or she performed a similar work in the past. In other words, the proficiency determination apparatus 1 can function as a system for training a worker.

### Fifth Embodiment

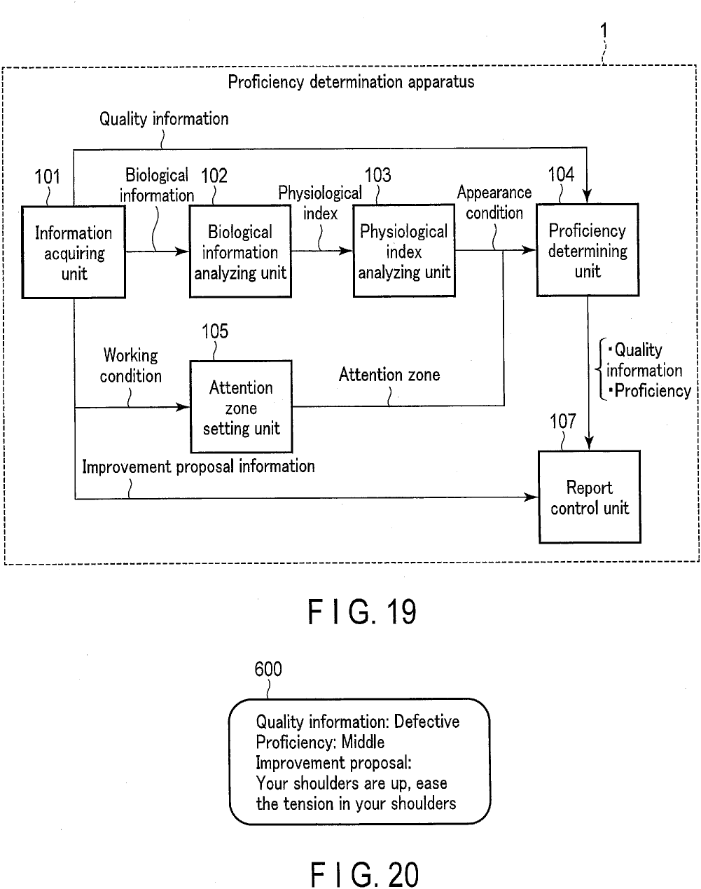

FIG. 19 is a block diagram showing a configuration example of a proficiency determination apparatus 1 according to the fifth embodiment. The proficiency determination apparatus 1 according to the fifth embodiment includes a report control unit 107 in addition to the components included in the proficiency determination apparatus 1 according to the third embodiment. The report control unit 107 is an example of a report controller. An information acquiring unit 101 according to the fifth embodiment is an example of a fifth acquiring unit.

The information acquiring unit 101 acquires improvement proposal information (advice) for a worker. More specifically, the information acquiring unit 101 acquires improvement proposal information for proposing a method of improving the action of a worker in a predetermined period. The improvement proposal information need only be data about the action of a worker performed in the period of data about biological information of the worker. For example, the improvement proposal information is an advice for the action of a worker in order to manufacture a high-quality product, or information indicating a check to be performed by the worker. The improvement proposal information can be information obtained by automatically analyzing image information showing the action of a worker by using a machine learning model or the like. Alternatively, the improvement proposal information can also be information manually input by a manager who manages a worker via an input interface 14 (see FIG. 21). Subsequently, the information acquiring unit 101 outputs the acquired improvement proposal information to the report control unit 107.

The report control unit 107 reports various kinds of information about the worker. For example, the report control unit 107 reports the improvement proposal information to the worker. Note that the report control unit 107 can operate so as to report no improvement proposal information if the proficiency of the worker is equal to or larger than a threshold, and report improvement proposal information if the proficiency of the worker is less than the threshold. Also, the report control unit 107 can operate so as to report no improvement proposal information if the quality information is good, and report improvement proposal information if the quality information is defective. Furthermore, when the quality information is defective, it is also possible to report no improvement proposal information if the proficiency of the worker is equal to or larger than the threshold, and report improvement proposal information if the proficiency of the worker is less than the threshold. Consequently, since an expert has already suffered a mental stress during the working time and recognizes that the product is defective, the report control unit 107 does not report improvement proposal information even though the quality information is defective. This makes it possible to prevent a further mental stress from being applied on the expert.

The report control unit 107 causes a display 12 or a speaker 13 (see FIG. 21) to report the improvement proposal information. The report control unit 107 can also report the improvement proposal information by, e.g., display data, a voice, a beep, or a vibration or contact of an object. In this case, the report control unit 107 can change the type of beep in accordance with whether the quality information is good or defective.

FIG. 20 is a view showing an example of the improvement proposal information according to the fifth embodiment. FIG. 20 shows report data 600 containing the quality information, the proficiency, and the improvement proposal information. The report data 600 indicates that the quality information is “defective”, the proficiency is “middle”, and the improvement proposal is “your shoulders are up, ease the tension in your shoulders”. The report data 600 can be displayed together with display data 510 and 520. Alternatively, the contents of the report data 600 can be output as a voice.

The proficiency determination apparatus 1 according to the fifth embodiment has been explained above. The proficiency determination apparatus 1 reports the improvement proposal information to a worker. Accordingly, the proficiency determination apparatus 1 can report points which the worker should be careful of in the next work. Consequently, the proficiency determination apparatus 1 can encourage the worker to spontaneously make efforts to improve the working technique based on the reported improvement proposal information in order to improve his or her proficiency. That is, the proficiency determination apparatus 1 can assist the worker to perform a work by becoming conscious of the quality of a product.

FIG. 21 is a block diagram showing a configuration example of hardware and software of the proficiency determination apparatus 1 according to the first to fifth embodiments. The proficiency determination apparatus 1 includes a processing circuit 10, a memory 11, a display 12, a speaker 13, an input interface 14, and a communication interface 15. These components are connected by a bus as a common signal transmission path such that they can communicate with each other. Each component need not be implemented by one hardware. For example, at least two components can also be implemented by one hardware.

The processing circuit 10 controls the operation of the proficiency determination apparatus 1. The processing circuit 10 includes a processor such as a CPU (Central Processing Unit), an MPU (Micro Processing Unit), or a CPU (Graphics Processing Unit) as hardware. The processing circuit 10 executes programs expanded on the memory 11 via the processor, thereby implementing the units (the information acquiring unit 101, the biological information analyzing unit 102, the physiological index analyzing unit 103, the proficiency determining unit 104, the attention zone setting unit 105, the display control unit 106, and the report control unit 107) corresponding to the individual programs. Note that each unit need not be implemented by the processing circuit 10 including a single processor. For example, each unit can also be implemented by the processing circuit 10 combining a plurality of processors.

The memory 11 stores information such as data and programs to be used by the processing circuit 10. The memory 11 includes a semiconductor memory element such as a RAM (Random Access Memory) as hardware. Note that the memory 11 can also be a driving device for reading and writing information with respect to an external storage device such as a magnetic disk (a floppy® disk or a hard disk), a magnetooptical disk (MO), an optical disk (a CD, a DVD, or a Blu-ray®) , a flash memory (a USB flash memory, a memory card, or an SSD), or a magnetic tape. Note that the storage area of the memory 11 can exist inside the proficiency determination apparatus 1 or in an external storage device. The memory 11 is an example of a storage unit.

The display 12 displays information such as data generated by the processing circuit 10 and data to be stored in the memory 11. As the display 12, it is possible to use a display such as a CRT (Cathode Ray Tube) display, an LCD (Liquid Crystal Display), a plasma display, an OELD (Organic Electro-Luminescence Display), or a tablet terminal. The display 12 can also display the display data. The display 12 is an example of a display unit or a report unit.

The speaker 13 reports information such as data generated by the processing circuit 10 and data to be stored in the memory 11 by a voice or a beep. The speaker 13 can also report the improvement proposal information. The speaker 13 is an example of a report unit.

The input interface 14 accepts input from the user of the proficiency determination apparatus 1, converts the accepted input into an electrical signal, and outputs the signal to the processing circuit 10. As the input interface 14, it is possible to use a physical manipulating component such as a mouse, a keyboard, a trackball, a switch, a button, a joystick, a touchpad, or a touch panel display. Note that the input interface 14 can also be a device that accepts input from an external input device different from the proficiency determination apparatus 1, converts the accepted input into an electrical signal, and outputs the signal to the processing circuit 10. The user is, e.g., a worker engaged in a technical work in a factory or the like. The input interface 14 is an example of an input unit.

The communication interface 15 transmits or receives data to and from an external device or an external network. An arbitrary communication standard can be used between the communication interface 15 and the external device or the external network. The communication method can be either a wired method or a wireless method. The proficiency determination apparatus 1 can transmit or receive data to or from a printer or the Internet, causes the printer to print out data generated by the proficiency determination apparatus 1, and display data on a web page, via the communication interface 15. The communication interface 15 is an example of a communication unit.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A proficiency determination apparatus comprising a processing circuit configured to:
acquire first time-series data about biological information of a worker in a predetermined period;
calculate second time-series data about a physiological index indicating a mental stress on the worker by analyzing the first time-series data;
calculate an appearance condition of the physiological index by analyzing the second time-series data; and
determine a proficiency of the worker based on the appearance condition of the physiological index.

2. The apparatus according to claim 1, wherein the processing circuit
acquires third time-series data about a working condition of the worker in the predetermined period,
sets an attention zone for the third time-series data, and
determines the proficiency of the worker based on the appearance condition of the physiological index in the attention zone.

3. The apparatus according to claim 2, wherein
the working condition is information indicating a working zone in which the worker is working during the predetermined period, a waiting zone in which the worker is waiting, and a working step zone in which the worker is executing a specific working step during the working zone, and
the processing circuit sets the working zone or the working step zone as the attention zone.

4. The apparatus according to claim 3, wherein the processing circuit
acquires quality information indicating quality of a product having undergone a work by the worker in the working zone,
sets, as the attention zone, a checking zone in which the worker is checking the quality of the product during the working zone, and
determines the proficiency of the worker based on the appearance condition of the physiological index and the quality information in the attention zone.

5. The apparatus according to claim 4, wherein
the quality information is information indicating whether the product is good or defective, and
the processing circuit determines the proficiency of the worker based on the appearance condition of the physiological index in the attention zone, when the product is defective.

6. The apparatus according to claim 1, wherein the processing circuit
acquires image information indicating an action of the worker in the predetermined period, and
causes a display to display display data obtained by relating at least one of the first time-series data, the second time-series data, the appearance condition of the physiological index, and the proficiency of the worker to the image information.

7. The apparatus according to claim 6, wherein the processing circuit causes the display to display the display data of a past work of the worker or another worker in relation to the display data of a current work of the worker.

8. The apparatus according to claim 1, wherein the processing circuit
acquires improvement proposal information for proposing a method of improving the action of the worker in the predetermined period, and
causes a reporting unit to report the improvement proposal information to the worker.

9. The apparatus according to claim 8, wherein the processing circuit does not cause the reporting unit to report the improvement proposal information when the proficiency of the worker is not less than a threshold, and causes the reporting unit to report the improvement proposal information when the proficiency of the worker is less than the threshold.

10. The apparatus according to claim 1, wherein the biological information or the physiological index is information about an NN interval, an electrodermal activity, perspiration, a blood flow, a body temperature, or a brain wave.

11. The apparatus according to claim 1, wherein the physiological index is an LF/HF ratio, an LF value, an HF value, a VLF value, a total power value, an HR value, a Mean NN value, an SDNN value, an RMSSD value, an NN50 value, a pNN50 value, or a CVRR value related to the NN interval.

12. The apparatus according to claim 1, wherein the appearance condition of the physiological index is a frequency or magnitude of a parameter including a local maximum, a local minimum, a mean, a dispersion, a fluctuation, a global maximum, a global minimum, a differential value, and an integral value related to the physiological index.

13. The apparatus according to claim 1, wherein the processing circuit determines the proficiency of the worker based on whether a frequency of the local maximum related to the physiological index is not less than a threshold.

14. A proficiency determination method comprising:
acquiring first time-series data about biological information of a worker in a predetermined period;
calculating second time-series data about a physiological index indicating a mental stress on the worker by analyzing the first time-series data;
calculating an appearance condition of the physiological index by analyzing the second time-series data; and
determining a proficiency of the worker based on the appearance condition of the physiological index.

15. A non-transitory computer readable medium including computer executable instructions, wherein the instructions, when executed by a processor, cause the processor to perform a method comprising:
acquiring first time-series data about biological information of a worker in a predetermined period;
calculating second time-series data about a physiological index indicating a mental stress on the worker by analyzing the first time-series data;
calculating an appearance condition of the physiological index by analyzing the second time-series data; and
determining a proficiency of the worker based on the appearance condition of the physiological index.
